(19) United States
(12) Patent Application Publication
(10) Pub. No.: US 2026/0364335 A1
(43) Pub. Date: Nov. 19, 2026

(54) BODILY FLUID FLOW ASSIST DEVICES AND METHODS TO IMPROVE SYSTEMIC CIRCULATION

(71) Applicant: Bard Peripheral Vascular, Inc., Franklin Lakes, NJ (US)

(72) Inventor: Summer L. FORD, Queen Creek, AZ (US)

(73) Assignee: Bard Peripheral Vascular, Inc., Franklin Lakes, NJ (US)

(21) Appl. No.: 19/102,106

(22) PCT Filed: Aug. 18, 2022
(86) PCT No.: PCT/US2022/075120

§ 371 Date: Feb. 7, 2025

(51) Int. Cl. A61M 60/226; A61M 60/126

(52) U.S. Cl. CPC A61M 60/226; A61M 60/126

(57) ABSTRACT

A bodily fluid flow assist device sized to be positioned at a junction between intersecting body conduits that includes a support body sized to engage an interior surface of a first body conduit of the intersecting body conduits. A first rotary wheel portion is rotatably connected within the support body and is configured to extend into a second body conduit of the intersecting body conduits with the support body engaged with the interior surface of the first body conduit. A second rotary wheel portion is operably connected to the first rotary wheel such that the second rotary wheel portion rotates with rotation of the first rotary wheel portion. The second rotary wheel portion is configured to be located at least partially within the first body conduit with the support body engaged with the interior surface of the first body conduit.

### TECHNICAL FIELD

The present specification generally relates to bodily fluid flow assist devices and methods to improve systemic circulation of bodily fluids.

### BACKGROUND

As cancer metastasizes from the original tumor into surrounding tissue, cancerous cells often become trapped within the lymph nodes of a patient. As a result, a physician may dissect one or more lymph nodes to thereby provide a diagnostic benefit of understanding whether or not the cancer has masticated from the surrounding tissue and to what extent. However, there may be certain effects associated with lymph node dissection including, but not limited to, leaving the patient with a predisposition for infection, lymphedema, oedema or seroma.

Current techniques for alieving lymphedema, oedema or seroma are physical massaging, exercise and compression devices. There is a need for additional methods and apparatuses for alieving lymphatic congestion.

### SUMMARY

According to a first embodiment, a bodily fluid flow assist device sized to be positioned at a junction between intersecting body conduits that includes a support body sized to engage an interior surface of a first body conduit of the intersecting body conduits. A first rotary wheel portion is rotatably connected within the support body and is configured to extend into a second body conduit of the intersecting body conduits with the support body engaged with the interior surface of the first body conduit. A second rotary wheel portion is operably connected to the first rotary wheel such that the second rotary wheel portion rotates with rotation of the first rotary wheel portion. The second rotary wheel portion is configured to be located at least partially within the first body conduit with the support body engaged with the interior surface of the first body conduit.

According to a second embodiment, a method of forming a bodily fluid flow assist device that is sized to be positioned in a first body conduit at a junction between the first body conduit and a second body conduit is provided. The method includes rotatably connecting a first rotary wheel portion within a support body such that at least a portion of the first rotary wheel portion is configured to extend into the second body conduit with the support body engaged with an interior surface of the first body conduit. The first rotary wheel portion is connected to a second rotary wheel portion such that the second rotary wheel portion rotates with rotation of the first rotary wheel portion. The second rotary wheel portion is configured to be located at least partially within the first body conduit with the support body engaged with the interior surface of the first body conduit.

According to a third embodiment, a method of increasing flow of lymphatic fluid from a lymphatic system toward a venous system of a patient, is provided. The method includes delivering a bodily fluid flow assist device sized to a first body conduit at junction between intersecting body conduits of the lymphatic system and the venous system. The flow assist device includes a support body sized and configured to engage an interior surface of the first body conduit. A first rotary wheel portion is rotatably connected within the support body and is configured to extend into a second body conduit of the intersecting body conduits with the support body engaged with the interior surface of the first body conduit. A second rotary wheel portion is operably connected to the first rotary wheel portion such that the second rotary wheel portion rotates with rotation of the first rotary wheel portion, the second rotary wheel portion configured to be located at least partially within the first body conduit with the support body engaged with the interior surface of the first body conduit. Blood flowing through the second body conduit rotates the first rotary wheel portion thereby rotating the second rotary wheel portion and pushing lymphatic fluid toward the second body conduit.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a bodily fluid flow assist device placed at a junction between the lymphatic system and the circulatory system, according to one or more embodiments shown and described herein;

FIG. 2 schematically depicts a side view of a rotary system for use in the fluid flow assist device of FIG. 1, according to one or more embodiments shown and described herein;

FIG. 3 schematically depicts an embodiment of a bodily fluid flow assist device, according to one or more embodiments shown and described herein;

FIG. 4 schematically depicts a side view of the fluid flow assist device of FIG. 3, according to one or more embodiments shown and described herein;

FIG. 5 schematically depicts another embodiment of a bodily fluid flow assist device, according to one or more embodiments shown and described herein;

FIG. 6 schematically depicts another embodiment of a bodily fluid flow assist device, according to one or more embodiments shown and described herein;

FIG. 7 schematically depicts a gear system of use in the fluid flow device of FIG. 6, according to one or more embodiments shown and described herein; and

FIG. 8 schematically depicts another embodiment of a bodily fluid flow assist device, according to one or more embodiments shown and described herein.

### DETAILED DESCRIPTION

Embodiments described herein are generally directed to a bodily fluid flow assist device sized to be positioned at a junction between intersecting body conduits, such as between a lymphatic duct and circulatory system. The fluid flow assist device includes a support body sized to engage an interior surface of a first body conduit of the intersecting body conduits. A first rotary wheel portion is rotatably connected within the support body and is configured to extend into a second body conduit of the intersecting body conduits with the support body engaged with the interior surface of the first body conduit. A second rotary wheel portion is operably connected to the first rotary wheel portion such that the second rotary wheel portion rotates with rotation of the first rotary wheel portion. The second rotary wheel portion is configured to be located at least partially within the first body conduit with the support body engaged with the interior surface of the first body conduit.

The fluid flow assist device includes a rotary system that includes the rotary wheel portions that is mounted to the covered support body that can be implanted between the lymphatic duct and the circulatory system at the left brachiocephalic vein (between the left subclavian and the left internal jugular veins) for improved circulation. The “cupped” or “scooped” design of the rotary wheel portions enable the rotary wheel portions to catch high pressure and velocity blood flow from the venous system, propelling the rotary wheel portions to rotate within the support body. While the rotary wheel portion is rotating on the blood flow side, the rotary wheel portion on the lymphatic duct side is forced to rotate, drawing fluid from the lymphatic duct out into the venous system, thereby decompressing the lymphatic system.

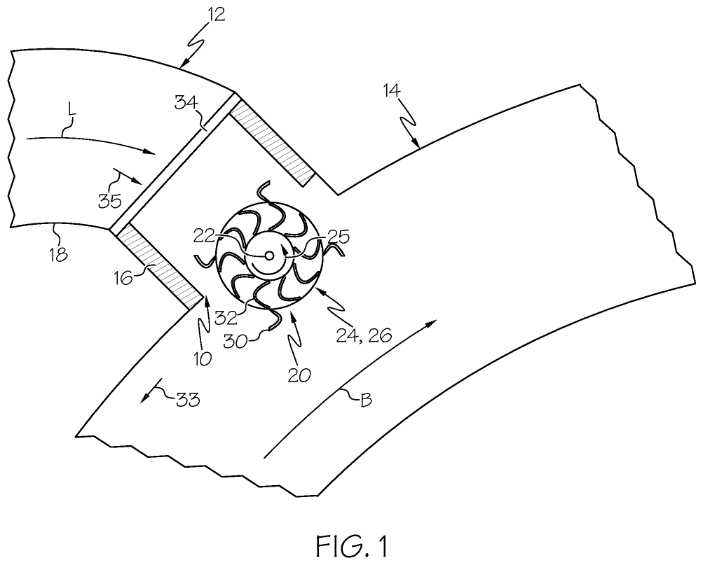

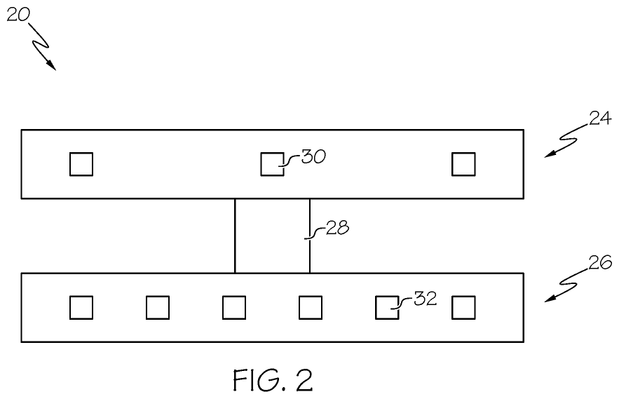

Referring to FIG. 1, a bodily fluid flow assist device 10 is schematically shown implanted at the entrance of the thoracic duct 12 into the left subclavian vein 14. The fluid flow assist device 10 includes a support body 16, such as a covered or uncovered expandable stent, that is engaged with a wall 18 of the thoracic duct 12. The support body 16 may, for example, be formed of a memory shape material that expands after being placed in the body. A rotary system, generally indicated as element 20, is rotatably connected to the support body 16 by a shaft thereby providing an axis of rotation 22. Referring also to FIG. 2, the rotary system 20 includes a first rotary wheel portion 24 that is operatively connected to a second rotary wheel portion 26. In the illustrated example, the first rotary wheel portion 24 is connected to the second rotary wheel portion 26 by a shaft 28; however, as will be described further below, other suitable connections between the first and the second rotary wheel portions 24 and 26 may be used.

The first rotary wheel portion 24 may be considered a drive wheel as the first rotary wheel portion 24 includes input paddles 30 that extend into the flow of blood B through the left subclavian vein 14. The input paddles 30 may be shaped, such as cupped toward an upstream direction 33 of the left subclavian vein 14 in order to catch energy from the impulse of the blood flowing by the fluid flow assist device 10. The impulse energy of the blood exerts a torque on the first rotary wheel portion 24, spinning the first rotary wheel portion 24 in the direction of arrow 25. Because the first rotary wheel portion 24 is connected to the second rotary wheel portion 26, rotation of the first rotary wheel portion 24 causes the second rotary wheel portion 26 to rotate. Because the rotation of the first rotary wheel portion 24 is driven by the high pressure venous flow, the fluid flow assist device 10 may be considered a passively-driven device in that there is not an actuator providing the driving force to the first rotary wheel portion 24.

The second rotary wheel portion 26 may be considered a driven wheel and includes output paddles 32 that extend into the flow of lymph through the thoracic duct 12. The output paddles 32 may be shaped, such as cupped toward a downstream direction of the thoracic duct 12 in the direction of arrow 35 in order to push lymph L into the blood flow through the higher pressure venous flow. The flow of the higher pressure venous flow is sufficient to overcome the resistance of the lymph flow through the thoracic duct 12 such that the output paddles 32 push the lymph toward the blood flow and decompress the lymphatic system. A one-way valve 34 may be provided at an entrance side of the support body 16 and connected thereto by any suitable connection. The one-way valve 34 may be formed of a flexible material that expands with the support body 16 and can allow the flow of lymph toward the left subclavian vein 14, but inhibit flow of blood into the lymphatic system.

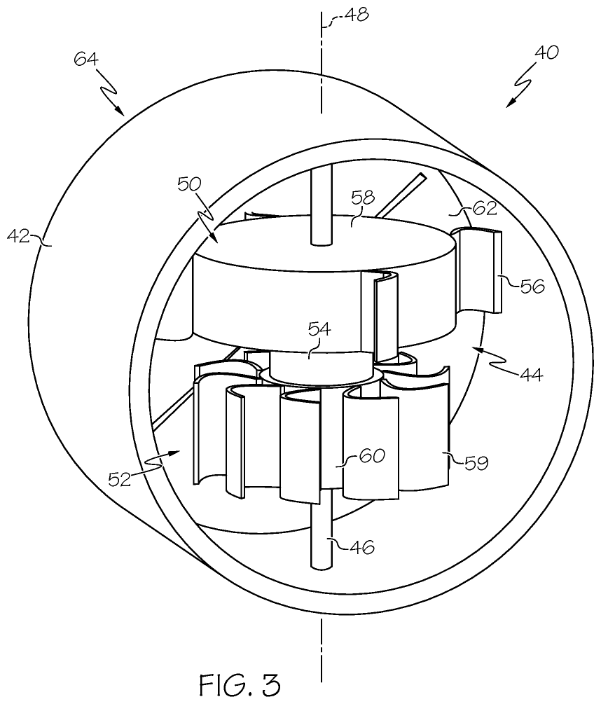

Referring now to FIG. 3, an embodiment of a fluid flow assist device 40 includes a support body 42 and rotary system 44 that is rotatably connected to the support body 42 by a shaft 46 thereby providing an axis of rotation 48 in a fashion similar to that described above. The rotary system 44 includes a first rotary wheel portion 50 that is operatively connected to a second rotary wheel portion 52 by a drive shaft 54.

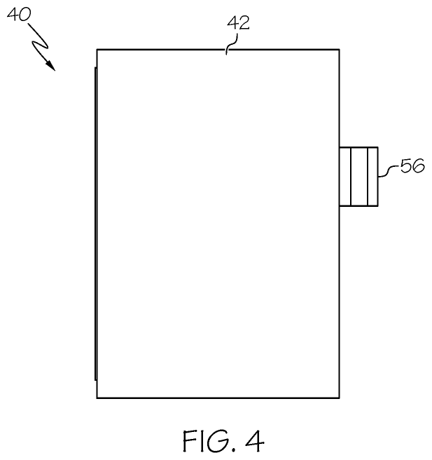

The first rotary wheel portion 50 includes input paddles 56 that are sized and shaped to extend into higher pressure venous flow. The input paddles 56 may be mounted about a periphery of a central hub wheel 58. In this example, referring also to FIG. 4, the central hub wheel 58 and the input paddles 56 are sized such that the input paddles 56 extend outside the support body 42 to catch the impulse energy from the blood flow. The second rotary wheel portion 52 includes output paddles 59 (FIG. 3). The output paddles 59 may be mounted about a periphery of a central hub wheel 60. The central hub wheel 60 has a reduced diameter compared to the central hub wheel 58 and the output paddles 59 are shaped and sized such that they do not extend outside the support body 42. The output paddles 59 are shaped and sized to push lymph from the lymphatic system and into the venous system. For example, there may be a greater number of the output paddles 59 than the input paddles 56 and the shapes, numbers of paddles and diameters of the hub wheels may be different to optimize rotation of the rotary system 44 to achieve a selected amount of rotation and lymph movement.

A one-way valve 62 may be placed at an entrance side 64 of the support body 42. The one-way valve 62 can allow the flow of lymph toward the venous system, but inhibit flow of blood into the lymphatic system.

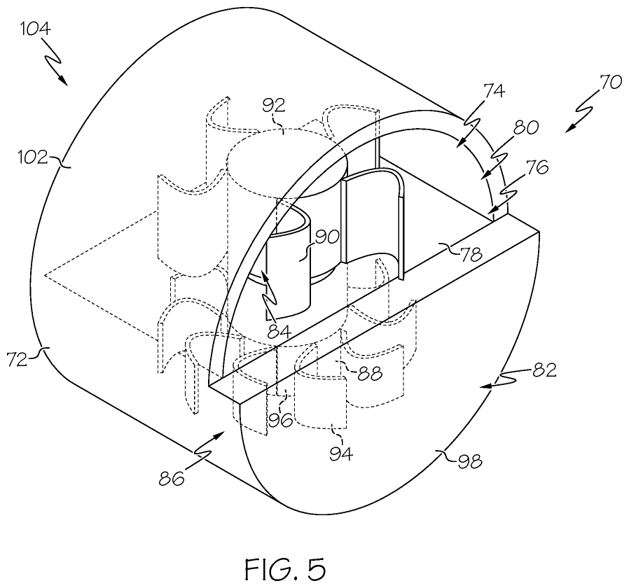

Referring to FIG. 5, another embodiment of a fluid flow assist device 70 is illustrated that includes a support body 72 and rotary system 74. In this embodiment, an inner volume 76 of the support body 72 is divided by a dividing wall 78 into a blood-contacting portion 80 and a lymph-contacting portion 82. The dividing wall 78 may be formed of a liquid impermeable hydrophobic or hydrophilic material and prevent passage of fluids between the blood-contacting portion 80 and the lymph-contacting portion 82.

The rotary system 74 may be rotatably connected to the support body 72 by the dividing wall 78, or may be rotatably connected to the support body 72 directly. The rotary system 74 includes a first rotary wheel portion 84 that is operatively connected to a second rotary wheel portion 86 by a drive shaft 88 that extends through the dividing wall 78. The first rotary wheel portion 84 includes input paddles 90 that are sized and shaped to extend into higher pressure venous flow. The input paddles 90 may be mounted about a periphery of a central hub wheel 92. The central hub wheel 92 and the input paddles 90 are sized such that the input paddles 90 extend outside the support body 72 to catch the impulse energy from the blood flow. The second rotary wheel portion 86 includes output paddles 94. The output paddles 94 may be mounted about a periphery of a central hub wheel 96. The output paddles 94 are shaped and sized such that they do not extend outside the support body 72. The output paddles 94 are shaped and sized to push lymph from the lymphatic system and into the venous system.

A one-way valve 98 may be placed at an exit side 100 of the lymph-contacting portion 82. The one-way valve 98 can allow the flow of lymph toward the venous system, but inhibit flow of blood into the lymphatic system. An isolation wall 102 may cover an entrance side 104 of the blood-contacting portion 80. The isolation wall 102 may be formed of a liquid impermeable hydrophobic or hydrophilic material and prevent passage of lymph into the blood-contacting portion 80 while allow the blood-contacting portion 80 exposure to the flow of blood.

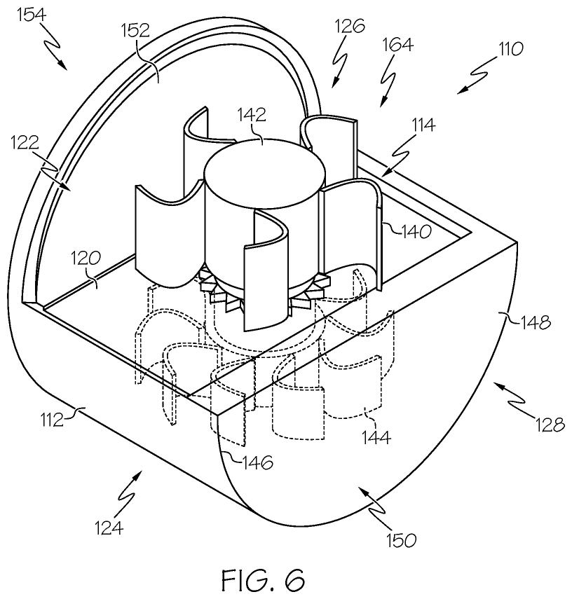

Referring to FIG. 6, another embodiment of a fluid flow assist device 110 is illustrated that includes a support body 112 and rotary system 114. In this embodiment, a lymph-contacting portion 124 is covered by the support body 112 while a blood-contacting portion 122 is uncovered by the support body 112. A dividing wall 120 divides the fluid flow assist device into the blood-contacting portion 122 and the lymph-contacting portion 124. The dividing wall 120 may be formed of a liquid impermeable material and prevent passage of fluids between the blood-contacting portion 122 and the lymph-contacting portion 124.

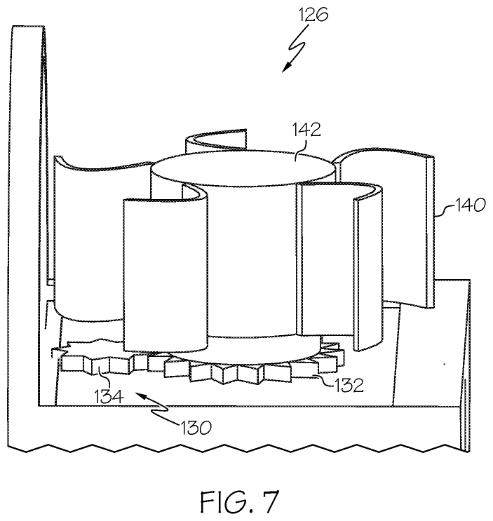

The rotary system 114 is rotatably connected to the support body 112 by the dividing wall 120. Referring also to FIG. 7, the rotary system 114 includes a first rotary wheel portion 126 that is operatively connected to a second rotary wheel portion 128. In this embodiment, the first rotary wheel portion 126 is connected to the second rotary wheel portion 128 by a gear system 130. For example, the first rotary wheel portion 126 may be connected to a first gear 132 having a first configuration and the second rotary wheel portion 128 may be connected to a second gear 134 having a second configuration that is meshed with teeth of the first gear 132. Because the first gear 132 is connected directly to the first rotary wheel portion 126 being rotated by the high pressure venous flow, it may be considered a drive gear and the second gear 134 may be considered a driven gear. A gear reduction can be provided when the drive gear is smaller in diameter and/or has fewer teeth than the driven gear. A gear overdrive can be provided when the drive gear is larger and/or has more teeth than the driven gear. Any of the embodiments described herein may include a gear train.

The first rotary wheel portion 126 includes input paddles 140 that are sized and shaped to extend into higher pressure venous flow. The input paddles 140 may be mounted about a periphery of a central hub wheel 142. The second rotary wheel portion 128 includes output paddles 144. The output paddles 144 may be mounted about a periphery of a central hub wheel 146 The output paddles 144 are shaped and sized to push lymph from the lymphatic system and into the venous system.

A one-way valve 148 may be placed at an exit side 150 of the lymph-contacting portion 124. The one-way valve 148 can allow the flow of lymph toward the venous system, but inhibit flow of blood into the lymphatic system. An isolation wall 152 may cover an entrance side 154 of the blood-contacting portion 122. The isolation wall 154 may be formed of a liquid impermeable hydrophobic or hydrophilic material and prevent passage of lymph into the blood-contacting portion 122.

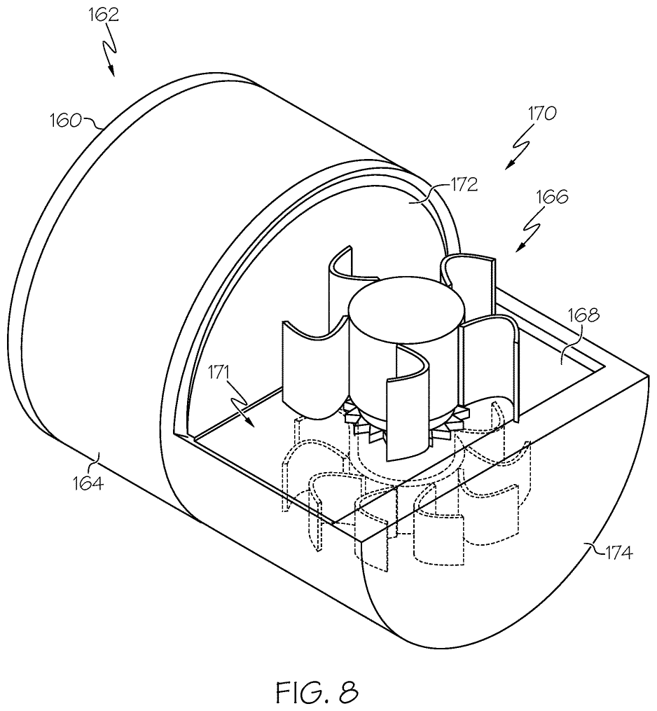

Referring now to FIG. 8, any of the flow assist devices disclosed herein may include a permeable flow regulation screen 160 at an entrance side 162 of a support body 164. In this example, a fluid flow device 170 includes the features described above with reference to flow assist device 110 including the support body 164, rotary system 166, dividing wall 168, gear train 171, isolation wall 172 and one-way valve 174. In this embodiment, the support body 164 extends outward from the isolation wall 172 and includes the flow regulation screen 160 covering the entrance side 162 of the support body 164. The flow regulation screen 160 can have a mesh size (number of holes per unit area) that is selected to regulate influx of lymph fluid when a negative pressure is induced inside the support body 164. The support body 164 provides stability and shape to inhibit lymph vessel collapse.

Any suitable materials may be used for forming the support bodies, regulation screens, walls and rotary members described herein. Examples of materials include biocompatible plastics and/or metals, such as stainless steel, titanium, cobalt chromium, platinum chromium and other alloys. Various coatings may also be used, such as sulfobetaine coating (tethered-liquid perfluorocarbon (TLP) surface), heparin-bonded collagen, polyethelennimine and heparin, amorphous silicone carbide, n-butyl methacrylate (PBMA)-poly(vinylidene fluoride-co-hexafluoropropylene) (PVDF-HFP) with everolimus drug, PVDF-HFP polymer with everolimus drug and phosphorylcholine (PC) polymer with zotarolimus drug.

The above-described flow assist devices can help negate the adverse effects following lymph node dissection by helping restore proper conduction of lymph fluid into the venous system. The flow assist devices are passive, taking advantage of the patient's own higher blood pressure of the venous system and helping to alleviate complications. The flow assist devices also overcome shortcomings of current surgical outcomes resulting from lymphaticovenular bypass surgeries, which are often subject to the problem of blood flowing backwards into the lymphatic system due to the high blood pressure/low lymphatic pressure dynamic.

Embodiments can be described with reference to the following numbered clauses, with certain features laid out in the dependent clauses:

Clause 1: A bodily fluid flow assist device sized to be positioned at a junction between intersecting body conduits, the assist device comprising: a support body sized to engage an interior surface of a first body conduit of the intersecting body conduits; a first rotary wheel portion rotatably connected within the support body and configured to extend into a second body conduit of the intersecting body conduits when the support body is engaged with the interior surface of the first body conduit; and a second rotary wheel portion operably connected to the first rotary wheel such that the second rotary wheel portion is configured to rotate with rotation of the first rotary wheel portion, the second rotary wheel portion configured to be located at least partially within the first body conduit when the support body is engaged with the interior surface of the first body conduit.

Clause 2: The assist device of clause 1, wherein the first rotary wheel portion and the second rotary wheel portion each comprise a plurality of paddles, wherein paddles of the second rotary wheel portion are configured to move a first bodily fluid flowing through the first body conduit toward the second body conduit and paddles of the first rotary wheel portion are configured to be moved by a first bodily fluid flowing through the second body conduit.

Clause 3: The assist device of clause 2, wherein the paddles of the first and second rotary wheel portions bend in opposite directions.

Clause 4: The assist device of any of clauses 1-3 further comprising a one-way valve that is connected to the support body, the one-way valve configured to block the second bodily fluid from flowing through the first body conduit.

Clause 5: The assist device of any of clauses 1-4 further comprising a dividing wall forming a first chamber that houses the first rotary wheel portion and a second chamber that houses the second rotary wheel portion.

Clause 6: The assist device of clause 5, wherein the dividing wall fluidly isolates the first chamber from the second chamber.

Clause 7: The assist device of clause 6 further comprising an isolation wall connected to the support body, the isolation wall configured to block flow of the first bodily fluid through the first chamber.

Clause 8: The assist device of clause 5-7 further comprising a one-way valve that is connected to the support body, the one-way valve configured to block flow of the second bodily fluid through the second chamber.

Clause 9: The assist device of clause 5-8 further comprising a flow regulation screen that covers at least a portion of an upstream end of the support body, the flow regulation screen configured to reduce flow of the first bodily fluid through the first chamber.

Clause 10: The assist device of any of clauses 1-9, wherein the assist device further comprises a shaft that connects the first rotary wheel portion to the second rotary wheel portion.

Clause 11: The assist device of clause 10, wherein the shaft is connected to one or more gears that change a rotational speed of the second rotary wheel portion relative to the first rotary wheel portion.

Clause 12: A method of forming a bodily fluid flow assist device that is sized to be positioned in a first body conduit at a junction between the first body conduit and a second body conduit, the method comprising: rotatably connecting a first rotary wheel portion within a support body such that at least a portion of the first rotary wheel portion is configured to extend into the second body conduit when the support body is engaged with an interior surface of the first body conduit; and connecting the first rotary wheel portion to a second rotary wheel portion such that the second rotary wheel portion is configured to rotate with rotation of the first rotary wheel portion, the second rotary wheel portion configured to be located at least partially within the first body conduit with the support body engaged with the interior surface of the first body conduit.

Clause 13: The method of clause 12, wherein the first rotary wheel portion and the second rotary wheel portion each comprise a plurality of paddles, wherein paddles of the second rotary wheel portion are configured to move a first bodily fluid flowing through the first body conduit toward the second body conduit and paddles of the first rotary wheel portion are configured to be moved by a second bodily fluid flowing through the second body conduit.

Clause 14: The method of clause 13, wherein the paddles of the first and second rotary wheels bend in opposite directions.

Clause 15: The method of clause 12 or 13 further comprising connecting a one-way valve to the support body, the one-way valve configured to block the second bodily fluid from flowing through the first body conduit.

Clause 16: The method of any of clauses 13-15 further comprising connecting a dividing wall to the support body, the dividing wall forming a first chamber that houses the first rotary wheel portion and a second chamber that houses the second rotary wheel portion.

Clause 17: The method of clause 16 further comprising connecting an isolation wall to the support body, the isolation wall configured to block flow of the first bodily fluid through the first chamber.

Clause 18: A method of increasing flow of lymphatic fluid from a lymphatic system toward a venous system of a patient, the method comprising: delivering a bodily fluid flow assist device sized to a first body conduit at junction between intersecting body conduits of the lymphatic system and the venous system, the assist device comprising: a support body sized and configured to engage an interior surface of the first body conduit; a first rotary wheel portion rotatably connected within the support body and configured to extend into a second body conduit of the intersecting body conduits with the support body engaged with the interior surface of the first body conduit; and a second rotary wheel portion operably connected to the first rotary wheel portion such that the second rotary wheel portion is configured to rotate with rotation of the first rotary wheel portion, the second rotary wheel portion configured to be located at least partially within the first body conduit when the support body is engaged with the interior surface of the first body conduit; and blood flowing through the second body conduit rotating the first rotary wheel portion thereby rotating the second rotary wheel portion and pushing lymphatic fluid toward the second body conduit.

Clause 19: The method of clause 18 further comprising inhibiting flow of blood from the second body conduit through the first body conduit using a one-way valve.

Clause 20: The method of clause 18 or 19 further comprising reducing a speed of rotation of the second rotary wheel relative to the first rotary wheel using one or more gears.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A bodily fluid flow assist device sized to be positioned at a junction between intersecting body conduits, the assist device comprising:
a support body sized to engage an interior surface of a first body conduit of the intersecting body conduits;
a first rotary wheel portion rotatably connected within the support body and configured to extend into a second body conduit of the intersecting body conduits when the support body is engaged with the interior surface of the first body conduit; and
a second rotary wheel portion operably connected to the first rotary wheel portion such that the second rotary wheel portion is configured to rotate with rotation of the first rotary wheel portion, the second rotary wheel portion configured to be located at least partially within the first body conduit when the support body engaged with the interior surface of the first body conduit.

2. The assist device of claim 1, wherein the first rotary wheel portion and the second rotary wheel portion each comprise a plurality of paddles, wherein paddles of the second rotary wheel portion are configured to move a first bodily fluid flowing through the first body conduit toward the second body conduit and paddles of the first rotary wheel portion are configured to be moved by a second bodily fluid flowing through the second body conduit.

3. The assist device of claim 2, wherein the paddles of the first and second rotary wheel portions bend in opposite directions.

4. The assist device of claim 1 further comprising a one-way valve that is connected to the support body, the one-way valve configured to block the second bodily fluid from flowing through the first body conduit.

5. The assist device of claim 1 further comprising a dividing wall forming a first chamber that houses the first rotary wheel portion and a second chamber that houses the second rotary wheel portion.

6. The assist device of claim 5, wherein the dividing wall fluidly isolates the first chamber from the second chamber.

7. The assist device of claim 6 further comprising an isolation wall connected to the support body, the isolation wall configured to block flow of the first bodily fluid through the first chamber.

8. The assist device of claim 5 further comprising a one-way valve that is connected to the support body, the one-way valve configured to block flow of the second bodily fluid through the second chamber.

9. The assist device of claim 5 further comprising a flow regulation screen that covers at least a portion of an upstream end of the support body, the flow regulation screen configured to reduce flow of the first bodily fluid through the first chamber.

10. The assist device of claim 1, wherein the assist device further comprises a shaft that connects the first rotary wheel to the second rotary wheel.

11. The assist device of claim 10, wherein the shaft is connected to one or more gears that change a rotational speed of the second rotary wheel relative to the first rotary wheel.

12. A method of forming a bodily fluid flow assist device that is sized to be positioned in a first body conduit at a junction between the first body conduit and a second body conduit, the method comprising:
rotatably connecting a first rotary wheel portion within a support body such that at least a portion of the first rotary wheel portion is configured to extend into the second body conduit when the support body is engaged with an interior surface of the first body conduit; and
connecting the first rotary wheel portion to a second rotary wheel portion such that the second rotary wheel portion is configured to rotate with rotation of the first rotary wheel portion, the second rotary wheel portion configured to be located at least partially within the first body conduit when the support body is engaged with the interior surface of the first body conduit.

13. The method of claim 12, wherein the first rotary wheel portion and the second rotary wheel portion each comprise a plurality of paddles, wherein paddles of the second rotary wheel are configured to move a first bodily fluid flowing through the first body conduit toward the second body conduit and paddles of the first rotary wheel are configured to be moved by a first bodily fluid flowing through the second body conduit.

14. The method of claim 13, wherein the paddles of the first and second rotary wheel portions bend in opposite directions.

15. The method of claim 12 further comprising connecting a one-way valve to the support body, the one-way valve configured to block the second bodily fluid from flowing through the first body conduit.

16. The method of claim 12 further comprising connecting a dividing wall to the support body, the dividing wall forming a first chamber that houses the first rotary wheel portion and a second chamber that houses the second rotary wheel portion.

17. The method of claim 16 further comprising connecting an isolation wall to the support body, the isolation wall configured to block flow of the first bodily fluid through the first chamber.

18. A method of increasing flow of lymphatic fluid from a lymphatic system toward a venous system of a patient, the method comprising:
delivering a bodily fluid flow assist device sized to a first body conduit at junction between intersecting body conduits of the lymphatic system and the venous system, the assist device comprising:
a support body sized and configured to engage an interior surface of the first body conduit;
a first rotary wheel portion rotatably connected within the support body and configured to extend into a second body conduit of the intersecting body conduits when the support body is engaged with the interior surface of the first body conduit; and
a second rotary wheel portion operably connected to the first rotary wheel portion such that the second rotary wheel portion is configured to rotate with rotation of the first rotary wheel portion, the second rotary wheel portion configured to be located at least partially within the first body conduit with the support body engaged with the interior surface of the first body conduit; and
blood flowing through the second body conduit rotating the first rotary wheel portion thereby rotating the second rotary wheel portion and pushing lymphatic fluid toward the second

19. The method of claim 18 further comprising inhibiting flow of blood from the second body conduit through the first body conduit using a one-way valve.

20. The method of claim 18 further comprising reducing a speed of rotation of the second rotary wheel relative to the first rotary wheel using one or more gears.
